(19) United States
(12) Patent Application Publication
(10) Pub. No.: US 2011/0060043 A1
(43) Pub. Date: Mar. 10, 2011

(54) INHIBITORS OF CELL PROLIFERATION AND USES THEREOF

(75) Inventors: George Reid, Heidelberg (DE); Maria Polycarpou-Schwarz, Heidelberg (DE); Frank Gannon, Dublin (IE)

(73) Assignee: European Molecular Biology Laboratory (EMBL), Heidelberg (DE)

(21) Appl. No.: 12/919,569

(22) PCT Filed: Mar. 2, 2009
(86) PCT No.: PCT/EP2009/001469

§ 371 Date: Nov. 22, 2010

Related U.S. Application Data
(60) Provisional application No. 61/032,293, filed on Feb. 28, 2008.

(51) Int. Cl. A61K 31/245; C07C 311/29; A61P 35/00; A61P 17/06; A61P 35/04

(52) U.S. Cl. 514/535; 560/12

(57) ABSTRACT

The present invention provides novel compounds that inhibit cell proliferation and uses of these compounds for treating, ameliorating or preventing diseases, conditions or disorders benefiting from the inhibition hyperproliferation.

The present invention provides novel compounds that inhibit cell proliferation, uses of these compounds for treating, ameliorating or preventing diseases, conditions or disorders benefiting from the inhibition of cell proliferation, in particular hyperproliferative diseases.

### BACKGROUND OF THE INVENTION

Proliferative diseases, in particular tumour diseases are among the leading causes of death in the developed world. While a large number of compounds are know that can inhibit aberrant cellular growth it has been found by the present inventors that certain phenylsulfonanilides are surprisingly potent inhibitors of cellular growth. Arylsulfonanilides have been used in a variety of non-medical and medical applications. In U.S. Pat. No. 6,265,142 the use of arylsulfonanilides as a component of a heat developable color photographic material further comprising a dye is disclosed. In this context the arylsulfonanilide serves the purpose of a developing agent. JP 2007 145738 A discloses that certain arylsulfonanilides act as hair growth inhibitor and can be included in cosmetic compositions. WO 2007/071443 discloses certain arylsulfonanilides and their use as inhibitors of CCR9 activity. It is taught that they are useful for therapeutic treatment of irritable bowel disease. WO 2006/04510 discloses certain arylsulfonanilides and their use in the treatment of cardiovascular diseases.

Some arylsulfonanilides are also known to have antiproliferative activity and one particular arylsulfonanilide-chloroindole termed, E7070, is currently in clinical trials for the treatment of cancer (Casini A. et al., (2002) Current Cancer Drug Targets 2:55-75). Further specific arylsulfonanilides, which have anti-tumor activity are taught, e.g. in Natarajan, A. et al. (2004) J. Medicinal Chem. 21: 4979-4982. These compounds were tested for their ability to inhibit the growth of the human lung cancer cell line A549. The phenylsulfonanilides with the highest anti-proliferative activity showed an IC₅₀ of 6 μM. Surprisingly, the phenylsulfonanilides of the present invention showed a significantly higher, e.g. up to about 40-fold higher, growth inhibitory potential than the arylsulfoanilides known from the prior art. Accordingly, the present invention provides novel phenylsulfonanilides, which are highly potent in the treatment of proliferative diseases.

### SUMMARY OF THE INVENTION

In a first embodiment the present invention relates to a compound of formula (I)

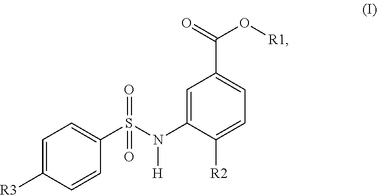

wherein
R¹ is C₁-C₆ alkyl;
R² is H, C₁-C₆ alkyl or C₁-C₆ alkoxy; and
R³ is H, C₁-C₆ alkyl C₁-C₆ alkoxy, or halogen;
under the proviso that R¹, R² and R³ do not have the following meaning:
(a) R¹ and R² are methyl and R³ is methyl, ethyl, n-propyl, iso-propyl, n-butyl, iso-butyl, methoxy, ethoxy, chloro, bromo, or fluoro;
(b) R¹ is ethyl, R² is methyl and R³ is methyl, methoxy, ethoxy, chloro, bromo, or fluoro;
(c) R¹ is methyl or ethyl, R² is H and R³ is H, methyl, ethyl, n-propyl, iso-propyl, n-butyl, iso-butyl, sec-butyl, tert-butyl, methoxy, ethoxy, chloro, bromo, or fluoro; or
(d) R¹ is n-propyl, R² is H and R³ methyl or chloro.

In a further embodiment the present invention relates to a pharmaceutical composition comprising a compound of the present invention or a compound according to formulas (II) to (III)

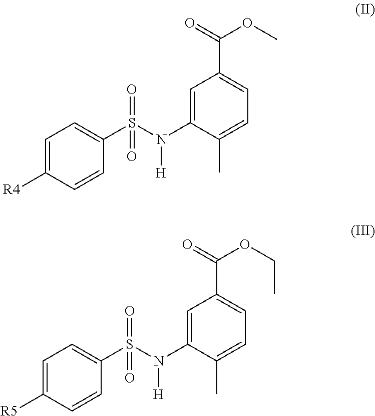

wherein
R⁴ is methyl, ethyl, n-propyl, iso-propyl, n-butyl, iso-butyl, methoxy, ethoxy, chloro, bromo, or fluoro; and
R⁵ is methyl, methoxy, ethoxy, chloro, bromo, or fluoro;
and one or more pharmaceutically acceptable excipient and/or carrier.

In a further embodiment the present invention relates to a compound of formula (I), wherein R¹ is C₁-C₆ alkyl; R² is H, C₁-C₆ alkyl, or C₁-C₆ alkoxy; and R³ is H, C₁-C₆ alkyl, C₁-C₆ alkoxy or halogen for use as a medicament.

In a further embodiment the present invention relates to a compound of formula (I), wherein R¹ is C₁-C₆ alkyl; R² is H, C₁-C₆ alkyl, or C₁-C₆ alkoxy; and R³ is H, C₁-C₆ alkyl, C₁-C₆ alkoxy, or halogen for treating, ameliorating or preventing a hyperproliferative disease.

In a further embodiment the present invention relates to a method of treating a hyperproliferative disease, wherein a pharmaceutically effective amount of a compound according to formula (I) to (XI), wherein in formula (I) R¹ is C₁-C₆ alkyl; R² is H, C₁-C₆ alkyl, or C₁-C₆ alkoxy; and R³ is H, C₁-C₆ alkyl, C₁-C₆ alkoxy, or halogen, in formulas (II) R⁴ is methyl, ethyl, n-propyl, iso-propyl, n-butyl, iso-butyl, methoxy, ethoxy, chloro, bromo, or fluoro, and in formula (III) R⁵ is methyl, ethyl, methoxy, ethoxy, chloro, bromo, or fluoro, is administered to a person in need thereof.

### DETAILED DESCRIPTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

In the following definitions of the terms: alkyl, alkoxy and halogen are provided. These terms will in each instance of its use in the remainder of the specification have the respectively defined meaning and preferred meanings. Nevertheless in some instances of their use throughout the specification preferred meanings of these terms are indicated.

The term “alkyl” refers to a saturated straight or branched carbon chain. Preferably, the chain comprises from 1 to 6 carbon atoms, i.e. 1, 2, 3, 4, 5, or 6, e.g. methyl, ethyl, n-propyl, iso-propyl, butyl, e.g. n-butyl, iso-butyl, sec-butyl, tert-butyl, pentyl, e.g. n-pentyl, sec-pentyl, iso-pentyl, tert-pentyl, neo-pentyl, hexyl, iso-hexyl, or n-hexyl.

The term “alkoxy” refers to an alkyl group linked to another group via an oxygen atom. Preferably, the alkyl chain of the alkoxy group comprises from 1 to 6 carbon atoms, i.e. 1, 2, 3, 4, 5, or 6 and, thus, preferred alkoxy groups are methoxy, ethoxy, n-propoxy, iso-propoxy iso-propoxy, butoxy, n-butoxy, iso-butoxy, sec-butoxy, tert-butoxy, n-pentoxy, sec-pentoxy, iso-pentoxy, tert-pentoxy, neo-pentoxy, hexoxy, iso-hexoxy, or n-hexoxy.

Compounds and also the starting materials for their preparation according to the invention can be synthesized by methods and standard procedures known to those skilled in the art, i.e. as described in the literature (for example in the standard works, such as Houben-Weyl, Methoden der organischen Chemie [Methods of Organic Chemistry], Georg-Thieme-Verlag, Stuttgart), to be precise under reaction conditions which are known to those skilled in the art and suitable for the said reactions. Use can also be made here of variants which are known per se, but are not mentioned here in greater detail.

Certain compounds of the present invention can exist in unsolvated forms as well as in solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (3H), iodine-125 (¹²⁵I) or carbon-14 (¹⁴C). All isotopic variations of the compounds of the present invention, whether radioactive or not, are intended to be encompassed within the scope of the present invention.

### Embodiments of the Invention

The present invention provides compounds of formula (I)

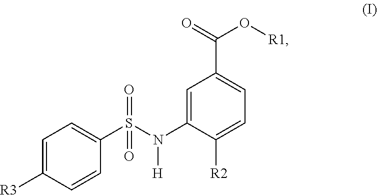

wherein
R¹ is C₁-C₆ alkyl, preferably C₁, C₂, C₃, C₄, C₅ or C₆ alkyl, e.g. methyl, ethyl, n-propyl, iso-propyl, butyl, e.g. n-butyl, iso-butyl, sec-butyl, tert-butyl, pentyl, e.g. n-pentyl, sec-pentyl, iso-pentyl, tert-pentyl, neo-pentyl, hexyl, iso-hexyl, or n-hexyl;
R² is H, C₁-C₆ alkyl, preferably C₁, C₂, C₃, C₄, C₅ or C₆ alkyl, e.g. methyl, ethyl, n-propyl, iso-propyl, butyl, e.g. n-butyl, iso-butyl, sec-butyl, tert-butyl, pentyl, e.g. n-pentyl, sec-pentyl, iso-pentyl, tert-pentyl, neo-pentyl, hexyl, iso-hexyl or n-hexyl or C₁-C₆ alkoxy, preferably C₁, C₂, C₃, C₄, C₅ or C₆ alkoxy, e.g. methoxy, ethoxy, n-propoxy, iso-propoxy iso-propoxy, butoxy, n-butoxy, iso-butoxy, sec-butoxy, tert-butoxy, n-pentoxy, sec-pentoxy, iso-pentoxy, tert-pentoxy, neo-pentoxy, hexoxy, iso-hexoxy, or n-hexoxy; and
R³ is H, C₁-C₆ alkyl, preferably C₁, C₂, C₃, C₄, C₅ or C₆ alkyl, e.g. methyl, ethyl, n-propyl, iso-propyl, butyl, e.g. n-butyl, iso-butyl, sec-butyl, tert-butyl, pentyl, e.g. n-pentyl, sec-pentyl, iso-pentyl, tert-pentyl, neo-pentyl, hexyl, iso-hexyl or n-hexyl; C₁-C₆ alkoxy, preferably C₁, C₂, C₃, C₄, C₅ or C₆ alkoxy, e.g. methoxy, ethoxy, n-propoxy, iso-propoxy iso-propoxy, butoxy, n-butoxy, iso-butoxy, sec-butoxy, tert-butoxy, n-pentoxy, sec-pentoxy, iso-pentoxy, tert-pentoxy, neo-pentoxy, hexoxy, iso-hexoxy, or n-hexoxy; or halogen; e.g. fluoro, chloro, bromo, or iodo;
   under the proviso that R¹, R² and R³ do not have the following meaning:
(a) R¹ and R² are methyl and R³ is methyl, ethyl, n-propyl, iso-propyl, n-butyl, iso-butyl, methoxy, ethoxy, chloro, bromo, or fluoro;
(b) R¹ is ethyl, R² is methyl and R³ is methyl, methoxy, ethoxy, chloro, bromo, or fluoro;
(c) R¹ is methyl or ethyl, R² is H and R³ is H, methyl, ethyl, n-propyl, iso-propyl, n-butyl, iso-butyl, sec-butyl, tert-butyl, methoxy, ethoxy, chloro, bromo, or fluoro; or
(d) R¹ is n-propyl, R² is H and R³ methyl or chloro.

In a preferred embodiment of the compound of the present invention R¹ is C₁-C₃ alkyl, preferably C₁, C₂, or C₃ alkyl, e.g. methyl, ethyl, n-propyl, iso-propyl and R² is H, or C₁-C₃ alkyl, preferably C₁, C₂, or C₃ alkyl, e.g. methyl, ethyl, n-propyl, or iso-propyl.

In a preferred embodiment of the compound of the present invention substituent R³ is methyl, ethyl, n-propyl, iso-propyl, methoxy, ethoxy, n-propoxy, iso-propoxy, F, Cl, or Br.

In a particularly preferred embodiment of the compound of the present invention R¹ is methyl, R² is n-propyl, or iso-propyl and R³ is methyl, ethyl, N-propyl, iso-propyl, methoxy, ethoxy, n-propoxy, iso-propoxy, F, Cl, or Br.

In a particularly preferred embodiment of the compound of the present invention R¹ is methyl, R² is H and R³ is n-propoxy, or iso-propoxy.

In a particularly preferred embodiment of the compound of the present invention R¹ is ethyl, R² is C₂ or C₃ alkyl, e.g. ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the compound of the present invention R¹ is ethyl, R² is H and R³ is n-propoxy or iso-propoxy.

In a particularly preferred embodiment of the compound of the present invention R¹ is iso-propyl, R² is H or C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the compound of the present invention R¹ is n-propyl, R² is C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the compound of the present invention R¹ is n-propyl, R² is H and R³ is ethyl, n-propyl, iso-propyl, methoxy, ethoxy, n-propoxy, iso-propoxy, F or Br.

In a particularly preferred embodiment of the compound of the present invention R¹ is methyl, R² is methyl, and R³ is n-propoxy or iso-propoxy.

In a particularly preferred embodiment of the compound of the present invention R¹ is methyl, R² ethyl and R³ is methyl, ethyl, n-propyl, iso-propyl, methoxy, ethoxy, n-propoxy, iso-propoxy, F, Cl, or Br.

In a particularly preferred embodiment of the compound of the present invention R¹ is methyl, R² is n-propyl and R³ is methyl, ethyl, n-propyl, iso-propyl, methoxy, ethoxy, n-propoxy, iso-propoxy, F, Cl, or Br.

In a particularly preferred embodiment of the compound of the present invention R¹ is methyl, R² is iso-propyl and R³ is methyl, ethyl, n-propyl, iso-propyl, methoxy, ethoxy, n-propoxy, iso-propoxy, F, Cl, or Br.

In a particularly preferred embodiment of the compound of the present invention R¹ is ethyl, R² is methyl and R³ is propoxy.

In a particularly preferred embodiment of the compound of the present invention R¹ is ethyl, R² is ethyl and R³ is methyl, ethyl, n-propyl, iso-propyl, methoxy, ethoxy, n-propoxy, iso-propoxy, F, Cl, or Br.

In a particularly preferred embodiment of the compound of the present invention R¹ is ethyl, R² is n-propyl and R³ is methyl, ethyl, n-propyl, iso-propyl, methoxy, ethoxy, n-propoxy, iso-propoxy, F, Cl, or Br.

In a particularly preferred embodiment of the compound of the present invention R¹ is ethyl, R² is iso-propyl and R³ is methyl, ethyl, n-propyl, iso-propyl, methoxy, ethoxy, n-propoxy, iso-propoxy, F, Cl, or Br.

In a particularly preferred embodiment of the compound of the present invention R¹ is n-propyl, R² is methyl and R³ is methyl, ethyl, n-propyl, iso-propyl, methoxy, ethoxy, n-propoxy, iso-propoxy, F, Cl, or Br.

In a particularly preferred embodiment of the compound of the present invention R¹ is n-propyl, R² is ethyl and R³ is methyl, ethyl, n-propyl, iso-propyl, methoxy, ethoxy, n-propoxy, iso-propoxy, F, Cl, or Br.

In a particularly preferred embodiment of the compound of the present invention R¹ is n-propyl, R² is n-propyl and R³ is methyl, ethyl, n-propyl, iso-propyl, methoxy, ethoxy, n-propoxy, iso-propoxy, F, Cl, or Br.

In a particularly preferred embodiment of the compound of the present invention R¹ is n-propyl, R² is iso-propyl and R³ is methyl, ethyl, n-propyl, iso-propyl, methoxy, ethoxy, n-propoxy, iso-propoxy, F, Cl, or Br.

In a particularly preferred embodiment of the compound of the present invention R¹ is iso-propyl, R² is methyl and R³ is methyl, ethyl, n-propyl, iso-propyl, methoxy, ethoxy, n-propoxy, iso-propoxy, F, Cl, or Br.

In a particularly preferred embodiment of the compound of the present invention R¹ is iso-propyl, R² is ethyl and R³ is methyl, ethyl, n-propyl, iso-propyl, methoxy, ethoxy, n-propoxy, iso-propoxy, F, Cl, or Br.

In a particularly preferred embodiment of the compound of the present invention R¹ is iso-propyl, R² is n-propyl and R³ is methyl, ethyl, n-propyl, iso-propyl, methoxy, ethoxy, n-propoxy, iso-propoxy, F, Cl, or Br.

In a particularly preferred embodiment of the compound of the present invention R¹ is iso-propyl, R² is iso-propyl and R³ is methyl, ethyl, n-propyl, iso-propyl, methoxy, ethoxy, n-propoxy, iso-propoxy, F, Cl, or Br.

In above indicated preferred embodiments of the compounds according to formula (I) the proviso similarly applies that R¹, R² and R³ do not have the following meaning:
(a) R¹ and R² are methyl and R³ is methyl, ethyl, n-propyl, iso-propyl, n-butyl, iso-butyl, methoxy, ethoxy, chloro, bromo, or fluoro;
(b) R¹ is ethyl, R² is methyl and R³ is methyl, methoxy, ethoxy, chloro, bromo, or fluoro;
(c) R¹ is methyl or ethyl, R² is H and R³ is H, methyl, ethyl, n-propyl, iso-propyl, n-butyl, iso-butyl, sec-butyl, tert-butyl, methoxy, ethoxy, chloro, bromo, or fluoro; or
(d) R¹ is n-propyl, R² is H and R³ methyl or chloro.

In a further aspect the present invention relates to a pharmaceutical composition comprising a compound of the present invention according to formula (I)

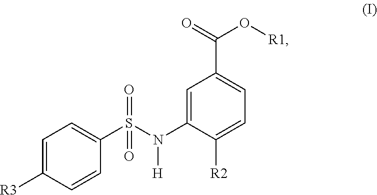

R¹ is C₁-C₆ alkyl, preferably C₁, C₂, C₃, C₄, C₅ or C₆ alkyl, e.g. methyl, ethyl, n-propyl, iso-propyl, butyl, e.g. n-butyl, iso-butyl, sec-butyl, tert-butyl, pentyl, e.g. n-pentyl, sec-pentyl, iso-pentyl, tert-pentyl, neo-pentyl, hexyl, iso-hexyl or n-hexyl;
R² is H, C₁-C₆ alkyl, preferably C₁, C₂, C₃, C₄, C₅ or C₆ alkyl, e.g. methyl, ethyl, n-propyl, iso-propyl, butyl, e.g. n-butyl, iso-butyl, sec-butyl, tert-butyl, pentyl, e.g. n-pentyl, sec-pentyl, iso-pentyl, tert-pentyl, neo-pentyl, hexyl, iso-hexyl or n-hexyl or C₁-C₆ alkoxy, preferably C₁, C₂, C₃, C₄, C₅ or C₆ alkoxy, e.g. methoxy, ethoxy, n-propoxy, iso-propoxy iso-propoxy, butoxy, n-butoxy, iso-butoxy, sec-butoxy, tert-butoxy, n-pentoxy, sec-pentoxy, iso-pentoxy, tert-pentoxy, neo-pentoxy, hexoxy, iso-hexoxy or n-hexoxy; and
R³ is H, C₁-C₆ alkyl, preferably C₁, C₂, C₃, C₄, C₅ or C₆ alkyl, e.g. methyl, ethyl, n-propyl, iso-propyl, butyl, e.g. n-butyl, iso-butyl, sec-butyl, tert-butyl, pentyl, e.g. n-pentyl, sec-pentyl, iso-pentyl, tert-pentyl, neo-pentyl, hexyl, iso-hexyl or n-hexyl; C₁-C₆ alkoxy, preferably C₁, C₂, C₃, C₄, C₅ or C₆ alkoxy, e.g. methoxy, ethoxy, n-propoxy, iso-propoxy iso-propoxy, butoxy, n-butoxy, iso-butoxy, sec-butoxy, tert-butoxy, n-pentoxy, sec-pentoxy, iso-pentoxy, tert-pentoxy, neo-pentoxy, hexoxy, iso-hexoxy or n-hexoxy; or halogen; e.g. F, Cl, Br, or I
   and one or more pharmaceutically acceptable excipient and/or carrier.

In a particular preferred embodiment the pharmaceutical composition comprises a compound having a structure according to formulas (II) or (III)

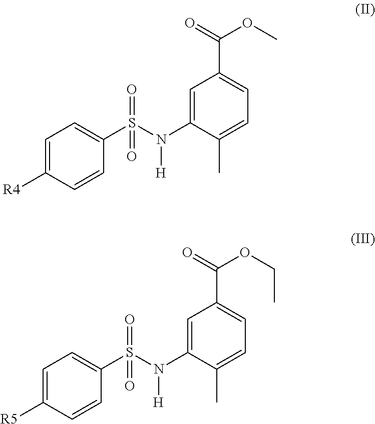

wherein
R⁴ is methyl, ethyl, n-propyl, iso-propyl, n-butyl, iso-butyl, methoxy, ethoxy, chloro, bromo, or fluoro, and
R⁵ is methyl, ethyl, methoxy, ethoxy, chloro, bromo, or fluoro,
   and one or more pharmaceutically acceptable excipient and/or carrier.

For preparing pharmaceutical compositions from the compounds of the present invention according to formula (I), in particular the compounds according to formula (II) to (III), pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired.

The powders and tablets preferably contain from 5% to 80%, more preferably from 20% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. Liquid forms are particularly preferred for topical applications to the eye. For parenteral injection, liquid preparations can be formulated in solution, like e.g. in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these, in packaged form.

It has been surprisingly found that a compound of formula (I), wherein

R¹ is C₁-C₆ alkyl;
R² is H, C₁-C₆ alkyl or C₁-C₆ alkoxy; and
R³ is H, C₁-C₆ alkyl C₁-C₆ alkoxy or halogen
has cytotoxic activity, in particular on tumor cells, and is, thus, suitable to be used as a medicament. Accordingly, in a further aspect the present invention relates to a compound of formula (I)

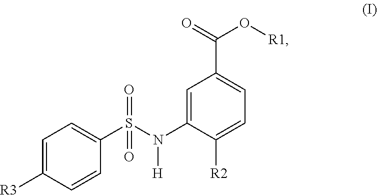

wherein
R¹ is C₁-C₆ alkyl, preferably C₁, C₂, C₃, C₄, C₅ or C₆ alkyl, e.g. methyl, ethyl, n-propyl, iso-propyl, butyl, e.g. n-butyl, iso-butyl, sec-butyl, tert-butyl, pentyl, e.g. n-pentyl, sec-pentyl, iso-pentyl, tert-pentyl, neo-pentyl, hexyl, iso-hexyl or n-hexyl;
R² is H, C₁-C₆ alkyl, preferably C₁, C₂, C₃, C₄, C₅ or C₆ alkyl, e.g. methyl, ethyl, n-propyl, iso-propyl, butyl, e.g. n-butyl, iso-butyl, sec-butyl, tert-butyl, pentyl, e.g. n-pentyl, sec-pentyl, iso-pentyl, tert-pentyl, neo-pentyl, hexyl, iso-hexyl or n-hexyl or C₁-C₆ alkoxy, preferably C₁, C₂, C₃, C₄, C₅ or C₆ alkoxy, e.g. methoxy, ethoxy, n-propoxy, iso-propoxy iso-propoxy, butoxy, n-butoxy, iso-butoxy, sec-butoxy, tert-butoxy, n-pentoxy, sec-pentoxy, iso-pentoxy, tert-pentoxy, neo-pentoxy, hexoxy, iso-hexoxy or n-hexoxy; and
R³ is H, C₁-C₆ alkyl, preferably C₁, C₂, C₃, C₄, C₅ or C₆ alkyl, e.g. methyl, ethyl, n-propyl, iso-propyl, butyl, e.g. n-butyl, iso-butyl, sec-butyl, tert-butyl, pentyl, e.g. n-pentyl, sec-pentyl, iso-pentyl, tert-pentyl, neo-pentyl, hexyl, iso-hexyl or n-hexyl; C₁-C₆ alkoxy, preferably C₁, C₂, C₃, C₄, C₅ or C₆ alkoxy, e.g. methoxy, ethoxy, n-propoxy, iso-propoxy iso-propoxy, butoxy, n-butoxy, iso-butoxy, sec-butoxy, tert-butoxy, n-pentoxy, sec-pentoxy, iso-pentoxy, tert-pentoxy, neo-pentoxy, hexoxy, iso-hexoxy or n-hexoxy; or halogen; e.g. F, Cl, Br, or I
   for use as a medicament.

In a preferred embodiment of the compound for use as a medicament R¹ is C₁-C₃ alkyl, preferably C₁, C₂, or C₃ alkyl, e.g. methyl, ethyl, n-propyl, iso-propyl and R² is H or C₁-C₃ alkyl, preferably C₁, C₂, or C₃ alkyl, e.g. methyl, ethyl, n-propyl, iso-propyl.

In a preferred embodiment of the compound for use as a medicament substituent R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is methyl, R² is H or C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is ethyl, R² is H or C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is n-propyl or iso-propyl, R² is H or C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is methyl, R² is H or C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is ethyl, R² is H or C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is n-propyl or iso-propyl, R² is H or C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is methyl, R² is methyl, and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is methyl, R² ethyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is methyl, R² is n-propyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is methyl, R² is iso-propyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is ethyl, R² is methyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is ethyl, R² is ethyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is ethyl, R² is n-propyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is ethyl, R² is iso-propyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is n-propyl, R² is methyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is n-propyl, R² is ethyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is n-propyl, R² is propyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is n-propyl, R² is iso-propyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is iso-propyl, R² is methyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is iso-propyl, R² is ethyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is iso-propyl, R² is propyl and R³ is methyl, ethyl, n-propyl, iso-propyl, methoxy, ethoxy, n-propoxy, iso-propoxy, F, Cl, or Br.

In a particularly preferred embodiment of the compound for use as a medicament R¹ is iso-propyl, R² is iso-propyl and R³ is methyl, ethyl, n-propyl, iso-propyl, methoxy, ethoxy, n-propoxy, iso-propoxy, F, Cl, or Br.

In particular preferred embodiments of the compound for use as a medicament, the compound has a structure according to formulas (IV) to (XI)

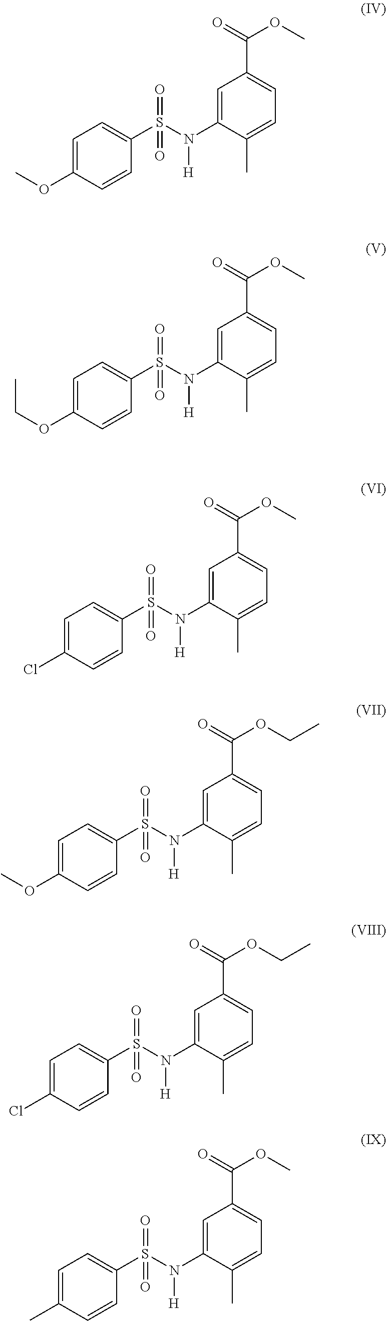

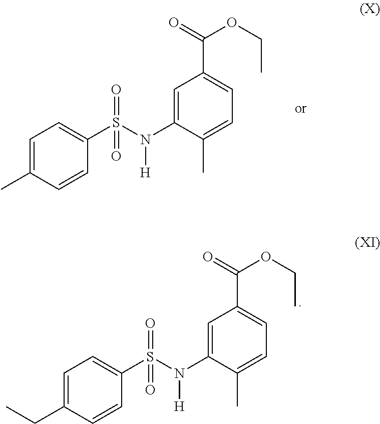

As has been indicated above the present invention is based in part on the discovery that the compounds for use as a medicament exert cytotoxicity on hyperproliferating cells as, e.g. the human tumor cell line MCF-7 (human breast cancer cell line). Accordingly, in a further embodiment the diseases that are treated with the compounds for use as a medicament are hyperproliferative disease. Thus the invention also relates to a compound of formula (I)

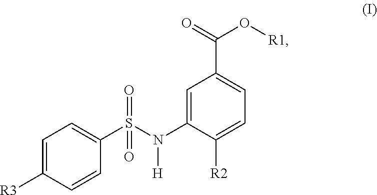

wherein
R¹ is C₁-C₆ alkyl, preferably C₁, C₂, C₃, C₄, C₅ or C₆ alkyl, e.g. methyl, ethyl, n-propyl, iso-propyl, butyl, e.g. n-butyl, iso-butyl, sec-butyl, tert-butyl, pentyl, e.g. n-pentyl, sec-pentyl, iso-pentyl, tert-pentyl, neo-pentyl, hexyl, e.g. iso-hexyl or n-hexyl;
R² is H, C₁-C₆ alkyl, preferably C₁, C₂, C₃, C₄, C₅ or C₆ alkyl, e.g. methyl, ethyl, n-propyl, iso-propyl, butyl, e.g. n-butyl, iso-butyl, sec-butyl, tert-butyl, pentyl, e.g. n-pentyl, sec-pentyl, iso-pentyl, tert-pentyl, neo-pentyl, hexyl, e.g. iso-hexyl or n-hexyl or C₁-C₆ alkoxy, preferably C₁, C₂, C₃, C₄, C₅ or C₆ alkoxy, e.g. methoxy, ethoxy, n-propoxy, iso-propoxy iso-propoxy, butoxy, n-butoxy, iso-butoxy, sec-butoxy, tert-butoxy, n-pentoxy, sec-pentoxy, iso-pentoxy, tert-pentoxy, neo-pentoxy, hexoxy, e.g. iso-hexoxy or n-hexoxy; and
R³ is H, C₁-C₆ alkyl, preferably C₁, C₂, C₃, C₄, C₅ or C₆ alkyl, e.g. methyl, ethyl, n-propyl, iso-propyl, butyl, e.g. n-butyl, iso-butyl, sec-butyl, tert-butyl, pentyl, e.g. n-pentyl, sec-pentyl, iso-pentyl, tert-pentyl, neo-pentyl, hexyl, e.g. iso-hexyl or n-hexyl; C₁-C₆ alkoxy, preferably C₁, C₂, C₃, C₄, C₅ or C₆ alkoxy, e.g. methoxy, ethoxy, n-propoxy, iso-propoxy iso-propoxy, butoxy, n-butoxy, iso-butoxy, sec-butoxy, tert-butoxy, n-pentoxy, sec-pentoxy, iso-pentoxy, tert-pentoxy, neo-pentoxy, hexoxy, e.g. iso-hexoxy or n-hexoxy; or halogen; e.g. F, Cl, Br, or I
   for treating, ameliorating or preventing a hyperproliferative disease.

In a preferred embodiment of the compound for treating, ameliorating or preventing a hyperproliferative disease R¹ is C₁-C₃ alkyl, preferably C₁, C₂, or C₃ alkyl, e.g. methyl, ethyl, n-propyl, iso-propyl and R² is H or C₁-C₃ alkyl, preferably C₁, C₂, or C₃ alkyl, e.g. methyl, ethyl, n-propyl, iso-propyl.

In a preferred embodiment of the compound for treating, ameliorating or preventing a hyperproliferative disease substituent R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is methyl, R² is H or C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is ethyl, R² is H or C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is propyl, R² is H or C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is methyl, R² is H or C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is ethyl, R² is H or C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is propyl, R² is H or C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is methyl, R² is methyl, and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is methyl, R² ethyl and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is methyl, R² is n-propyl and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is methyl, R² is iso-propyl and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is ethyl, R² is methyl and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is ethyl, R² is ethyl and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is ethyl, R² is n-propyl and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is ethyl, R² is iso-propyl and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is n-propyl, R² is methyl and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is n-propyl, R² is ethyl and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is n-propyl, R² is n-propyl and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is n-propyl, R² is iso-propyl and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is iso-propyl, R² is methyl and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is iso-propyl, R² is ethyl and R³ is methyl, ethyl, n-propyl, iso-propyl, methoxy, ethoxy, n-propoxy, iso-propoxy, F, Cl, or Br.

In a particularly preferred embodiment of the compound for treating, ameliorating or preventing a hyperproliferative disease R¹ is iso-propyl, R² is n-propyl and R³ is methyl, ethyl, n-propyl, iso-propyl, methoxy, ethoxy, n-propoxy; iso-propoxy, F, Cl, or Br.

In a particularly preferred embodiment of the compound for treating, ameliorating or preventing a hyperproliferative disease R¹ is iso-propyl, R² is iso-propyl and R³ is methyl, ethyl, n-propyl, iso-propyl, methoxy, ethoxy, n-propoxy, iso-propoxy, F, Cl, or Br.

In particular preferred embodiments of the compound for treating, ameliorating or preventing a hyperproliferative disease, the compound has a structure according to formulas (IV) to (XI)

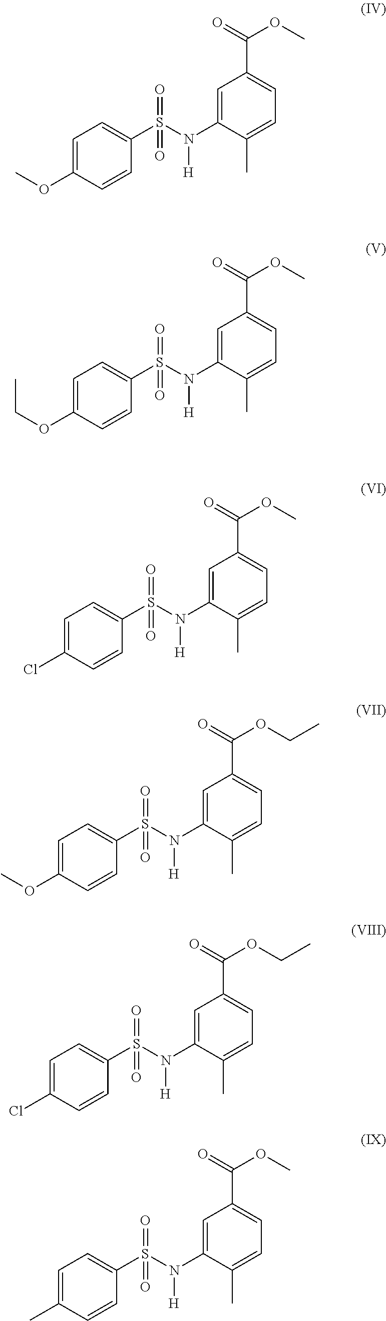

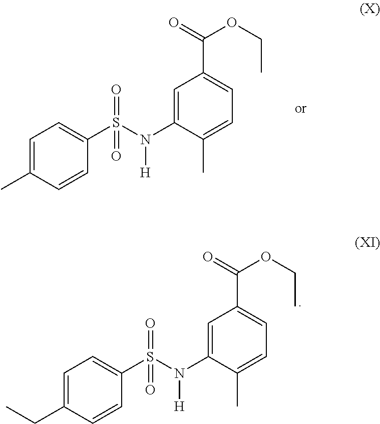

Similarly, in a further embodiment the present invention relates to a method of treating a hyperproliferative disease, wherein a compound of formula (I)

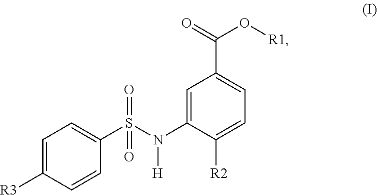

wherein
R¹ is C₁-C₆ alkyl, preferably C₁, C₂, C₃, C₄, C₅ or C₆ alkyl, e.g. methyl, ethyl, n-propyl, iso-propyl, butyl, e.g. n-butyl, iso-butyl, sec-butyl, tert-butyl, pentyl, e.g. n-pentyl, sec-pentyl, iso-pentyl, tert-pentyl, neo-pentyl, hexyl, e.g. iso-hexyl or n-hexyl;
R² is H, C₁-C₆ alkyl, preferably C₁, C₂, C₃, C₄, C₅ or C₆ alkyl, e.g. methyl, ethyl, n-propyl, iso-propyl, butyl, e.g. n-butyl, iso-butyl, sec-butyl, tert-butyl, pentyl, e.g. n-pentyl, sec-pentyl, iso-pentyl, tert-pentyl, neo-pentyl, hexyl, e.g. iso-hexyl or n-hexyl or C₁-C₆ alkoxy, preferably C₁, C₂, C₃, C₄, C₅ or C₆ alkoxy, e.g. methoxy, ethoxy, n-propoxy, iso-propoxy iso-propoxy, butoxy, n-butoxy, iso-butoxy, sec-butoxy, tert-butoxy, n-pentoxy, sec-pentoxy, iso-pentoxy, tert-pentoxy, neo-pentoxy, hexoxy, e.g. iso-hexoxy or n-hexoxy; and
R³ is H, C₁-C₆ alkyl, preferably C₁, C₂, C₃, C₄, C₅ or C₆ alkyl, e.g. methyl, ethyl, n-propyl, iso-propyl, butyl, e.g. n-butyl, iso-butyl, sec-butyl, tert-butyl, pentyl, e.g. n-pentyl, sec-pentyl, iso-pentyl, tert-pentyl, neo-pentyl, hexyl, e.g. iso-hexyl or n-hexyl; C₁-C₆ alkoxy, preferably C₁, C₂, C₃, C₄, C₅ or C₆ alkoxy, e.g. methoxy, ethoxy, n-propoxy, iso-propoxy iso-propoxy, butoxy, n-butoxy, iso-butoxy, sec-butoxy, tert-butoxy, n-pentoxy, sec-pentoxy, iso-pentoxy, tert-pentoxy, neo-pentoxy, hexoxy, e.g. iso-hexoxy or n-hexoxy; or halogen; e.g. F, Cl, Br, or I or of a composition of the present invention is administered to a patient in need thereof. It is preferred that the compound is administered in a pharmaceutically effective amount.

In a preferred embodiment the composition further comprises one or more cytotoxic and/or cytostatic compounds. Preferred examples of such compounds are provided below.

In a preferred embodiment of the method of treating, ameliorating or preventing a hyperproliferative disease R¹ is C₁-C₃ alkyl, preferably C₁, C₂, or C₃ alkyl, e.g. methyl, ethyl, n-propyl, iso-propyl and R² is H or C₁-C₃ alkyl, preferably C₁, C₂, or C₃ alkyl, e.g. methyl, ethyl, propyl, iso-propyl.

In a preferred embodiment of the method of treating, ameliorating or preventing a hyperproliferative disease R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is methyl, R² is H or C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is ethyl, R² is H or C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is propyl, R² is H or C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is methyl, R² is H or C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is ethyl, R² is H or C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is propyl, R² is H or C₁-C₃ alkyl, preferably C₁, C₂, or C₃ alkyl, e.g. methyl, ethyl, n-propyl, iso-propyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is methyl, R² is methyl, and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is methyl, R² ethyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is methyl, R² is propyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is methyl, R² is iso-propyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is ethyl, R² is methyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is ethyl, R² is ethyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is ethyl, R² is n-propyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is ethyl, R² is iso-propyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is n-propyl, R² is methyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is n-propyl, R² is ethyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is n-propyl, R² is n-propyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is n-propyl, R² is iso-propyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is iso-propyl, R² is methyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is iso-propyl, R² is ethyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is iso-propyl, R² is n-propyl and R³ is methyl, ethyl, n-propyl, iso-propyl, methoxy, ethoxy, n-propoxy, iso-propoxy, F, Cl, or Br.

In a particularly preferred embodiment of the method of treating, ameliorating or preventing a hyperproliferative disease R¹ is iso-propyl, R² is iso-propyl and R³ is methyl, ethyl, n-propyl, iso-propyl, methoxy, ethoxy, n-propoxy, iso-propoxy, F, Cl, or Br.

In particular preferred embodiment of the method of treating, ameliorating or preventing a hyperproliferative disease, the compound has a structure according to formulas (IV) to (XI)

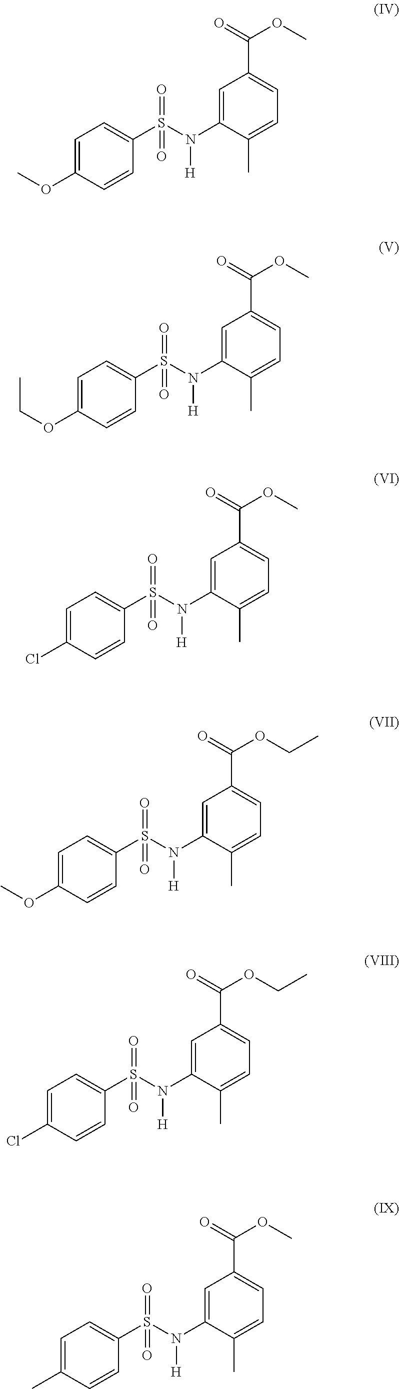

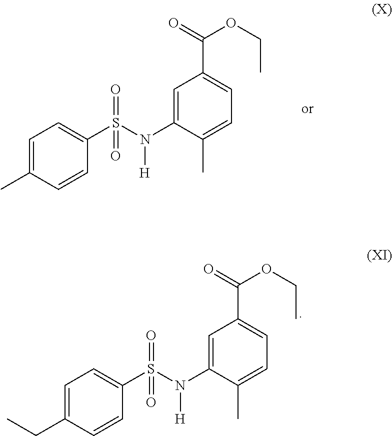

It is further preferred that the hyperproliferative diseases are selected from the group consisting of precancerosis; dysplasia; metaplasia; carcinomas of the gastrointestinal or colorectal tract, liver, pancreas, kidney, bladder, prostate, endometrium, ovary, testes, melanoma, dysplastic oral mucosa, invasive oral cancers, small cell and non-small cell lung carcinomas, hormone-dependent breast cancers, hormone-independent breast cancers, transitional and squamous cell cancers, neurological malignancies including neuroblastoma, gliomas, astrocytomas, osteosarcomas, soft tissue sarcomas, hemangioamas, endocrinological tumors, hematologic neoplasias and cancers, including leukemias, lymphomas, and other myeloproliferative and lymphoproliferative diseases, carcinomas in situ, hyperplastic lesions, adenomas, fibromas, histiocytosis, chronic inflammatory proliferative diseases, vascular proliferative diseases and virus-induced proliferative diseases, skin diseases characterized by hyperproliferation of keratinocytes and/or T cells. The present inventors have found that particular preferred diseases treatable with the compounds of the present invention are haematological cancers, in particular leukemia, lymphomas, and myelomas; breast cancer, in particular hormone-dependent breast cancers, or hormone-independent breast cancers; or lung cancer, in particular small cell or non-small cell lung carcinomas.

The precancerosis treatable with the compounds of the present invention are preferably selected from the group consisting of precancerosis of the skin, in particular actinic keratosis, cutaneaous horn, actinic cheilitis, tar keratosis, arsenic keratosis, x-ray keratosis, Bowen's disease, bowenoid papulosis, lentigo maligna, lichen sclerosus, and lichen rubber mucosae; precancerosis of the digestive tract, in particular erythroplakia, leukoplakia, Barrett's esophagus, Plummer-Vinson syndrome, crural ulcer, gastropathia hypertrophica gigantea, borderline carcinoma, neoplastic intestinal polyp, rectal polyp, porcelain gallbladder; gynaecological precancerosis, in particular carcinoma ductale in situ (CDIS), cervical intraepithelial neoplasia (CIN), leukoplakia, endometrial hyperplasia (grade III), vulvar dystrophy, vulvar intraepithelial neoplasia (VIN), hydatidiform mole; urologic precancerosis, in particular bladder papillomatosis, Queyrat's erythroplasia, testicular intraepithelial neoplasia (TIN), leukoplakia; carcinoma in situ (CIS); precancerosis caused by chronic inflammation, in particular pyoderma, osteomyelitis, acne conglobata, lupus vulgaris, and fistula.

Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exist chronic irritation or inflammation. Dysplastic disorders which can be treated with the compounds of the present invention include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis heminelia, dysplasia epiphysialis multiplex, dysplasia epiphysalis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia hidrotic ectodermal dysplasia, hypohidrotic ectodermal dysplasia, lymphopenic thymic dysplasia, mammary dysplasia, mandibulofacial dysplasia, metaphysical dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, ophthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Metaplasia is a form of controlled cell growth in which one type of adult or fully differentiated cell substitutes for another type of adult cell. Metaplastic disorders, which are treatable are preferably selected from the group consisting of agnogenic myeloid metaplasia, apocrine metaplasia, atypical metaplasia, autoparenchymatous metaplasia, connective tissue metaplasia, epithelial metaplasia, intestinal metaplasia, metaplastic anemia, metaplastic ossification, metaplastic polyps, myeloid metaplasia, primary myeloid metaplasia, secondary myeloid metaplasia, squamous metaplasia, squamous metaplasia of amnion, symptomatic myeloid metaplasia and regenerative metaplasia.

Many skin diseases are characterized by hyperproliferation of keratinocytes and/or T cells. Examples of such diseases which are treatable with the compounds of the present invention comprise without limitations psoriasis in particular psoriasis vulgaris, psoriasis capitis, psoriasis guttata, psoriasis inversa; neurodermatitis; ichtyosises; alopecia greata; alopecia totalis; alopecia subtotalis; alopecia universalis; alopecia diffusa; atopic dermatitis; lupus erythematodes of the skin; dermatomyositis of the skin; atopic eczema; morphea; scleroderma; alopecia greata Ophiasis type; androgenic alopecia; allergic contact dermatitis; irritative contact dermatitis; contact dermatitis; pemphigus vulgaris; pemphigus foliaceus; pemphigus vegetans; scarring mucous membrane pemphigoid; bullous pemphigoid; mucous membrane pemphigoid; dermatitis; dermatitis herpetiformis Duhring; urticaria; necrobiosis lipoidica; erythema nodosum; prurigo simplex; prurigo nodularis; prurigo acuta; linear IgA dermatosis; polymorphic light dermatosis; erythema solaris; exanthema of the skin; drug exanthema; purpura chronica progressiva; dihydrotic eczema; eczema; fixed drug exanthema; photoallergic skin reaction; and periorale dermatitis.

The quantity of active component in a unit dose preparation administered in the use of the present invention may be varied or adjusted from about 1 mg to about 1000 mg per m², preferably about 5 mg to about 150 mg/m² according to the particular application and the potency of the active component. The pharmaceutical composition can, if desired, in a combination therapy also contain other compatible therapeutic agents known to have antiproliferative activity. Thus, in a further aspect the present invention is directed at a pharmaceutical composition comprising

a compound of formula (I)

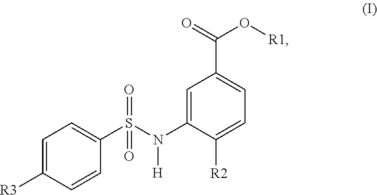

wherein
R¹ is C₁-C₆ alkyl, preferably C₁, C₂, C₃, C₄, C₅ or C₆ alkyl, e.g. methyl, ethyl, n-propyl, iso-propyl, butyl, e.g. n-butyl, iso-butyl, sec-butyl, tert-butyl, pentyl, e.g. n-pentyl, sec-pentyl, iso-pentyl, tert-pentyl, neo-pentyl, hexyl, e.g. iso-hexyl or n-hexyl;
R² is H, C₁-C₆ alkyl, preferably C₁, C₂, C₃, C₄, C₅ or C₆ alkyl, e.g. methyl, ethyl, n-propyl, iso-propyl, butyl, e.g. n-butyl, iso-butyl, sec-butyl, tert-butyl, pentyl, e.g. n-pentyl, sec-pentyl, iso-pentyl, tert-pentyl, neo-pentyl, hexyl, e.g. iso-hexyl or n-hexyl or C₁-C₆ alkoxy, preferably C₁, C₂, C₃, C₄, C₅ or C₆ alkoxy, e.g. methoxy, ethoxy, n-propoxy, iso-propoxy iso-propoxy, butoxy, n-butoxy, iso-butoxy, sec-butoxy, tert-butoxy, n-pentoxy, sec-pentoxy, iso-pentoxy, tert-pentoxy, neo-pentoxy, hexoxy, e.g. iso-hexoxy or n-hexoxy; and
R³ is H, C₁-C₆ alkyl, preferably C₁, C₂, C₃, C₄, C₅ or C₆ alkyl, e.g. methyl, ethyl, n-propyl, iso-propyl, butyl, e.g. n-butyl, iso-butyl, sec-butyl, tert-butyl, pentyl, e.g. n-pentyl, sec-pentyl, iso-pentyl, tert-pentyl, neo-pentyl, hexyl, e.g. iso-hexyl or n-hexyl; C₁-C₆ alkoxy, preferably C₁, C₂, C₃, C₄, C₅ or C₆ alkoxy, e.g. methoxy, ethoxy, n-propoxy, iso-propoxy iso-propoxy, butoxy, n-butoxy, iso-butoxy, sec-butoxy, tert-butoxy, n-pentoxy, sec-pentoxy, iso-pentoxy, tert-pentoxy, neo-pentoxy, hexoxy, e.g. iso-hexoxy or n-hexoxy; or halogen; e.g. F, Cl, Br, or I
   and one or more cytotoxic and/or cytostatic compound, preferably a pharmaceutically effective amount of the compound of formula (I). These compounds preferably include, but are not limited to, pure or mixed anti-estrogens such as faslodex, tamoxifen or raloxifen; any inhibitors of topoisomerase I or II, such as camptothecin (topo I) or etoposide (topo II); any compound that acts through inhibiting aromatase activity, such as anastrozole or letrozole; any preparation that interferes with HER2 or HER3 signalling such as herceptin; any compound that interchelates DNA, such as doxorubicin. Particularly preferred cytostatic or cytotoxic drugs, which can be combined with the compounds of the present invention are alkylating substances, anti-metabolites, antibiotics, epothilones, nuclear receptor agonists and antagonists, anti-androgenes, anti-estrogens, platinum compounds, hormones and antihormones, interferons and inhibitors of cell cycle-dependent protein kinases (CDKs), inhibitors of cyclooxygenases and/or lipoxygenases, biogeneic fatty acids and fatty acid derivatives, including prostanoids and leukotrienes, inhibitors of protein kinases, inhibitors of protein phosphatases, inhibitors of lipid kinases, platinum coordination complexes, ethyleneimenes, methylmelamines, trazines, vinca alkaloids, pyrimidine analogs, purine analogs, alkylsulfonates, folic acid analogs, anthracendiones, substituted urea, methylhydrazin derivatives, in particular acediasulfone, aclarubicine, ambazone, aminoglutethimide, L-asparaginase, azathioprine, bleomycin, busulfan, calcium folinate, carboplatin, carpecitabine, carmustine, celecoxib, chlorambucil, cis-platin, cladribine, cyclophosphamide, cytarabine, dacarbazine, dactinomycin dapsone, daunorubicin, dibrompropamidine, diethylstilbestrole, docetaxel, doxorubicin, enediynes, epirubicin, epothilone B, epothilone D, estramucin phosphate, estrogen, ethinylestradiole, etoposide, flavopiridol, floxuridine, fludarabine, fluorouracil, fluoxymesterone, flutamide fosfestrol, furazolidone, gemcitabine, gonadotropin releasing hormone analog, hexamethylmelamine, hydroxycarbamide, hydroxymethylnitrofurantoin, hydroxyprogesteronecaproat, hydroxyurea, idarubicin, idoxuridine, ifosfamide, interferon α, irinotecan, leuprolide, lomustine, lurtotecan, mafenide sulfate olamide, mechlorethamine, medroxyprogesterone acetate, megastrolacetate, melphalan, mepacrine, mercaptopurine, methotrexate, metronidazole, mitomycin C, mitopodozide, mitotane, mitoxantrone, mithramycin, nalidixic acid, nifuratel, nifuroxazide, nifuralazine, nifurtimox, nimustine, ninorazole, nitrofurantoin, nitrogen mustards, oleomucin, oxolinic acid, pentamidine, pentostatin, phenazopyridine, phthalylsulfathiazole, pipobroman, prednimustine, prednisone, preussin, procarbazine, pyrimethamine, raltitrexed, rapamycin, rofecoxib, rosiglitazone, salazosulfapyridine, scriflavinium chloride, semustine streptozocine, sulfacarbamide, sulfacetamide, sulfachlopyridazine, sulfadiazine, sulfadicramide, sulfadimethoxine, sulfaethidole, sulfafurazole, sulfaguanidine, sulfaguanole, sulfamethizole, sulfamethoxazole, co-trimoxazole, sulfamethoxydiazine, sulfamethoxypyridazine, sulfamoxole, sulfanilamide, sulfaperin, sulfaphenazole, sulfathiazole, sulfisomidine, staurosporin, tamoxifen, taxol, teniposide, tertiposide, testolactone, testosteronpropionate, thioguanine, thiotepa, timidazole, topotecan, triaziquone, treosulfan, trimethoprim, trofosfamide, UCN-01, vinblastine, vincristine, vindesine, vinblastine, vinorelbine, and zorubicin, or their respective derivatives or analogs thereof. Several of the above indicated drugs are now administered simultaneously for cancer therapy and, consequently, it is also envisioned that more than one cytostatic and/or cytotoxic drug is comprised in compositions of the present invention.

In therapeutic use the compounds of the present invention, in particular the compounds according to formula (II) to (XI) are administered at an initial dosage of about 0.05 mg/kg to about 20 mg/kg daily. A daily dose range of about 0.05 mg/kg to about 2 mg/kg is preferred, with a daily dose range of about 0.05 mg/kg to about 1 mg/kg being most preferred. The dosages, however, may be varied depending upon the requirements of the patient, the severity of the condition being treated, and the compound being employed. Determination of the proper dosage for a particular situation is within the skill of the practitioner. Generally, treatment is initiated with smaller dosages, which are less than the optimum dose of the compound. Thereafter, the dosage is increased by small increments until the optimum effect under circumstances is reached. For convenience, the total daily dosage may be divided and administered in portions during the day, if desired.

Synthesis of the Compounds

The compounds of the general formula (I) according to the present invention may be prepared according to the following scheme I:

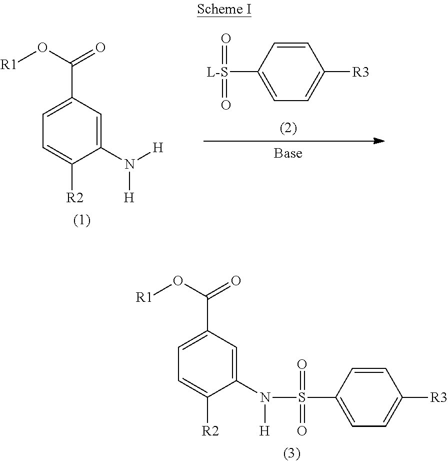

wherein R¹ is C₁-C₆ alkyl; R² is H, C₁-C₆ alkyl or C₁-C₆ alkoxy; and R³ is H, C₁-C₆ alkyl C₁-C₆ alkoxy or halogen or have the particularly preferred meanings as defined above, L is a conventional leaving group such as halogen or the like or is an activating group for the sulfonyl group, i.e. an activated ester. The above process comprises reacting a compound of the genera formula (1) with a sulfonyl group providing agent with a leaving group L of the general formula (2) in an organic solvent to obtain a compound of the general formula (3). The reaction may be carried out in a conventional organic solvent such as, for example, tetrahydrofuran, dichloromethane, acetonitrile, chloroform and dimethylformamide, preferably dichloromethane.

And also the reaction is preferably carried out in the presence of a coupling agent such as a conventional inorganic or an organic base.

Such conventional inorganic or organic bases used in the reaction may include sodium hydride, potassium hydride, sodium hydroxide, potassium hydroxide, sodium carbonate, potassium carbonate, cesium carbonate, sodium bicarbonate, potassium bicarbonate, triethylamine, pyridine and DBU, preferably pyridine.

The reaction may be carried out at a temperature between 3° C. and boiling point of the solvent used, preferably at 50° C.-100° C. and for 5-48 hours, preferably for 10-24 hours.

In the above reaction process, if any acid material is formed, a basic material may be added as a scavenger in order to eliminate the acid material from the reaction phase. Such basic material may be alkali metal hydroxide, alkali earth metal hydroxide, alkali metal oxide, alkali earth metal oxide, alkali metal carbonate, alkali earth metal carbonate, alkali metal hydrogen carbonate, alkali earth metal hydrogen carbonate such as for example, sodium hydroxide, potassium hydroxide, calcium hydroxide, magnesium hydroxide, magnesium oxide, calcium oxide, potassium carbonate, sodium carbonate, calcium carbonate, magnesium carbonate, magnesium bicarbonate, sodium bicarbonate, calcium bicarbonate or the like, and organic amines, or corresponding polymer bound basic scavengers.

The reaction products may be separated from any non-reacted educts and/or from impurities by several purification methods known in the art. Preferred methods include HPLC.

In a preferred embodiment a substituted sulfonylchloride (2) (1.5 eq.) is dissolved in dichloromethane at a concentration of 0.1 mol/L, pyridine (3 eq.) is added and the reaction solution is stirred. Subsequently, 3-amino-4-alkyl-benzoate or 3-amino-4-alkoxy-benzoate (1) is added (1 eq.) at room temperature and stirring is continued for 18 hours. The reaction mixture is purified via preparative HPLC.

### Experimental Section

### 1. Synthesis and Analysis of Preferred Compounds

### 1.1 Synthesis of 3-[[(4-methylphenyl)sulfonyl]amino]-4-methyl-benzoic acid methyl ester (Compound (IX))

Reagents and solvents were obtained from commercial suppliers and were used without further purification. ¹H NMR spectra were recorded on a DPX-250 Bruker Avance spectrometer—chemical shifts are reported as δ (ppm) downfield from tetramethylsilane as internal standard. HPLC data were obtained using an Agilent 1100 HPLC system under the following conditions:

### Column: Luna C18 3 μm, 30×2.0 mm.

### Temperature: 40° C.

Mobile phase: A=0.05% Trifluoroacetic acid in water.
B=0.05% Trifluoroacetic acid in acetonitrile.
   Flow rate: 1.0 ml/min.
   Gradient: 95% A/5% B to 5% A/95% B over 4 min, then hold for 1.5 min.
   Sample detection: UV at 215 nm.

Toluene-4-sulfonyl chloride (60.3 mmol, 11.5 g) was added in portions at 0° C. over 10 minutes to a stirred solution of methyl 3-amino-4-methylbenzoate (60.5 mmol, 10.0 g) in anhydrous pyridine (215 cm³) under an atmosphere of nitrogen. The mixture was allowed to warm to ambient temperature and stirring was continued for 24 hours. After this time, the reaction mixture was poured onto ice/water (500 cm³) and the product was extracted into ethyl acetate (3×200 cm³). The combined organic extracts were washed with 2 M hydrochloric acid (7×100 cm³), water (100 cm³), saturated aqueous sodium hydrogen carbonate solution (100 cm3) and brine (100 cm³), then dried (MgSO₄) and filtered. The solvents were removed in vacuo and the residue was triturated with hexane (10 cm3). The resulting solid was collected by filtration and dried in vacuo at 40° C. to give methyl 3-[[(4-methylphenyl)sulfonyl]amino]-4-methyl-benzoic acid methyl ester (16.08 g, 83.5%) as an off-white solid. 1H NMR (CDCl₃) δ 2.1 (s, 3H, ArCH3), 2.4 (s, 3H, ArCH₃), 3.9 (s, 3H, CO₂CH₃), 6.6 (s, 1H, NH), 7.1 (d, 1H, J=8 Hz, ArH), 7.2 (d, 2H, J=8 Hz, ArH), 7.6 (d, 2H, J=8 Hz, ArH), 7.7 (dd, 1H, J=8 Hz, J=2 Hz, ArH), 7.9 (d, 1H, J=2 Hz, ArH). HPLC 100% at 3.27 min.

### 1.2 Analysis of 3-[[(4-methoxyphenyl)sulfonyl]amino]-4-methyl-benzoic acid ethyl ester (Compound (VII))

Molecular formula: C₁₇H₁₉NO₅S having a theoretical molecular weight of 349.41. Measured on HPLC/MS* using a X-bridge C₁₈-column, 5 μm particle size, 4.6×150 mm (diameter×length) at a flow rate of 1.75 mL/min with a linear gradient of water to acetonitril from initially 99:1 to 1:99 (after 9.10 min) and using a 3100 Mass detector from Waters. Apparent mass was MH⁺=350 at t=7.21 min. NMR spectra were recorded using a Bruker UltraShield Advance 400 (400 MHz, 1H; 100 MHz) spectrometer and calibrated using residual undeuterated solvent as an internal reference. ¹H NMR (CDCl₃) δ 1.4 (q, 3H, J=7 Hz, CH₂CH₃), 2.1 (s, 3H, ArCH₃), 3.8 (s, 3H, OMe), 4.4 (t, 2H, J=7 Hz, CH₂CH₃), 6.4 (s, 1H, NH), 6.9 (d, 2H, J=9 Hz, ArH), 7.2 (d, 1H, J=8 Hz, ArH), 7.7 (d, 2H, J=9 Hz, ArH), 7.8 (d, 1H, J=8 Hz, ArH), 7.9 (s, 1H, ArH).

### 1.3 Analysis of 3-[[(4-chlorophenyl)sulfonyl]amino]-4-methyl-benzoic acid ethyl ester (Compound (VIII))

Molecular formula: C₁₆H₁₆ClNO₄S having a theoretical molecular weight of 353.83. Measured on HPLC/MS* using a X-bridge C₁₈-column, 5 μm particle size, 4.6×150 mm (diameter×length) at a flow rate of 1.75 mL/min with a linear gradient of water to acetonitril from initially 99:1 to 1:99 (after 9.10 min) and using a 3100 Mass detector from Waters. Apparent mass was MH⁺=354 at t=7.81 min. NMR spectra were recorded using a Bruker UltraShield Advance 400 (400 MHz, 1H; 100 MHz) spectrometer and calibrated using residual undeuterated solvent as an internal reference. ¹H NMR (CDCl₃) δ 1.4 (q, 3H, J=7 Hz, CH₂CH₃), 2.1 (s, 3H, ArCH₃), 4.4 (t, 2H, J=7 Hz, CH₂CH₃), 6.4 (s, 1H, NH), 7.2 (d, 1H, J=8 Hz, ArH), 7.5 (d, 2H, J=9 Hz, ArH), 7.7 (d, 2H, J=9 Hz, ArH), 7.8 (d, 1H, J=8 Hz, ArH), 7.9 (s, 1H, ArH).

### 1.4 Analysis of 3-[[(4-methylphenyl)sulfonyl]amino]-4-methyl-benzoic acid ethyl ester (Compound (X))

Molecular formula: C₁₇H₁₉NO₄S having a theoretical molecular weight of 333.41. Measured on HPLC/MS* using a X-bridge C₁₈-column, 5 μm particle size, 4.6×150 mm (diameter×length) at a flow rate of 1.75 mL/min with a linear gradient of water to acetonitril from initially 99:1 to 1:99 (after 9.10 min) and using a 3100 Mass detector from Waters. Apparent mass was MH⁺=334 at t=7.53 min. NMR spectra were recorded using a Bruker UltraShield Advance 400 (400 MHz, 1H; 100 MHz) spectrometer and calibrated using residual undeuterated solvent as an internal reference. ¹H NMR (CDCl₃) δ 1.4 (q, 3H, J=7 Hz, CH₂CH₃), 2.1 (s, 3H, ArCH₃), 2.4 (s, 3H, ArCH₃), 4.3 (t, 2H, J=7 Hz, CH₂CH₃), 6.4 (br s, 1H, NH), 7.2 (d, 1H, J=8 Hz, ArH), 7.3 (d, 2H, J=8 Hz, ArH), 7.7 (d, 2H, J=8 Hz, ArH), 7.8 (d, 1H, J=8 Hz, ArH), 7.9 (s, 1H, ArH).

### 1.5 Summary of Analysis of Molecular Masses and Retention Times of Compounds (IV) to (XI)

Retention time was measured on HPLC/MS using a Waters X-bridge C₁₈-column, 5 μm particle size, 4.6×150 mm (diameter×length) at a flow rate of 1.75 mL/min with a linear gradient (water to acetonitrile, 0.2% formic acid as modifier) from initially 99:1 to 1:99 over 9.10 min, then hold for 1.80 min. Mass signals were measured using a Waters 3100 Mass detector. The results for compound (IV) to (XI) are shown in Table 1 below.

TABLE 1

| | | mass | retention time |
|---|---|---|---|
| | Compound name | (MH⁺) | (min., LC-MS) |
| (IV) | 3-[[(4-methoxyphenyl)sulfonyl]amino]- | 336 | 6.81 |
| | 4-methyl-benzoic acid methyl ester | | |
| (V) | 3-[[(4-ethoxyphenyl)sulfonyl]amino]-4- | 350 | 7.26 |
| | methyl-benzoic acid methyl ester | | |
| (VI) | 3-[[(4-chlorophenyl)sulfonyl]amino]-4- | 340 | 7.41 |
| | methyl-benzoic acid methyl ester | | |
| (VII) | 3-[[(4-methoxyphenyl)sulfonyl]amino]- | 350 | 7.21 |
| | 4-methyl-benzoic acid ethyl ester | | |
| (VIII) | 3-[[(4-chlorophenyl)sulfonyl]amino]-4- | 354 | 7.81 |
| | methyl-benzoic acid ethyl ester | | |
| (IX) | 3-[[(4-methylphenyl)sulfonyl]amino]-4- | 320 | 7.12 |
| | methyl-benzoic acid methyl ester | | |
| (X) | 3-[[(4-methylphenyl)sulfonyl]amino]-4- | 334 | 7.53 |
| | methyl-benzoic acid ethyl ester | | |
| (XI) | 3-[[(4-ethylphenyl)sulfonyl]amino]-4- | 348 | 7.95 |
| | methyl-benzoic acid ethyl ester | | |

### 2. Cytotoxicity Assay

Preferred compounds were analyzed for their ability to inhibit growth of tumour cells (e.g. MCF7, HL60, LL2, HeLa, PC-3, A549 and A375 cells) in vitro. Growth inhibition was tested at various concentrations in a 10 point two fold serial dilution and cells were incubated under standard mammalian tissue culture conditions for 72 hours in triplicate. Cell viability was measured by measuring ATP levels in viable cells using the ATPLite kit (PerkinElmer) as described in the user manual. Raw data was transformed to percentage inhibition of growth compared to a DMSO only control and values were expressed as IC₅₀ in micromolar calculated using XLfit (IDBS). The compounds of the present invention showed the ability to inhibit the growth of various tumor cell lines indicating a broad spectrum of growth inhibitory activity The results obtained when using compounds (IV), (V), (VI) and (IX) on MCF-7 cells are depicted in Table 2 below

TABLE 2

| Compound | IC₅₀ in MCF-7 cells in μM |
|---|---|
| Compound (IV) | 0.23 |
| Compound (V) | 0.16 |
| Compound (VI) | 1.10 |
| Compound (IX) | 0.28 |

1. Compound of formula (I) 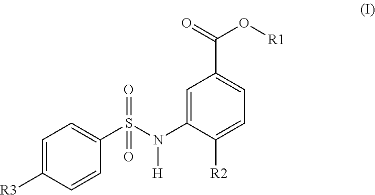
wherein
R¹ is Ci-C₆ alkyl;
R² is H, C₁-C₆ alkyl or C₁-C₆ alkoxy; and
R³ is H, C₁-C₆ alkyl C₁-C₆ alkoxy or halogen;
under the proviso that R¹, R² and R³ do not have the following meaning:
(a) R¹ and R² are methyl and R³ is methyl, ethyl, n-propyl, iso-propyl, n-butyl, iso-butyl, methoxy, ethoxy, chloro, bromo, or fluoro;
(b) R¹ is ethyl, R² is methyl and R³ is methyl, methoxy, ethoxy, chloro, bromo, or fluoro;
(c) R¹ is methyl or ethyl, R² is H and R³ is H, methyl, ethyl, n-propyl, iso-propyl, H-butyl, iso-butyl, sec-butyl, tert-butyl, methoxy, ethoxy, chloro, bromo, or fluoro; or
(d) R¹ is n-propyl, R² is H and R³ methyl or chloro.

2. Compound of claim 1, wherein
R¹ is C₁-C₃ alkyl; and
R² is H or C₁-C₃ alkyl.

3. Compound of claim 1, wherein
R³ is a methyl, ethyl, n-propyl, methoxy, ethoxy, n-propoxy, isopropoxy, Fluoro, chloro or bromo.

4. Composition comprising a compound 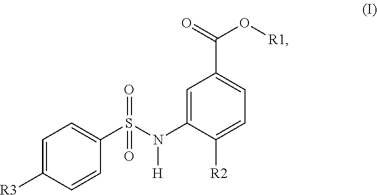
wherein R¹ is C₁-C₆ alkyl;
R² is H, C₁-C₆ alkyl or C₁-C₆ alkoxy; and
R³ is H, C₁-C₆ alkyl C₁-C₆ alkoxy or halogen;
and one or more pharmaceutically acceptable excipient and/or carrier.

5. Composition according to claim 4 having a structure according to formulas (II) or (III) 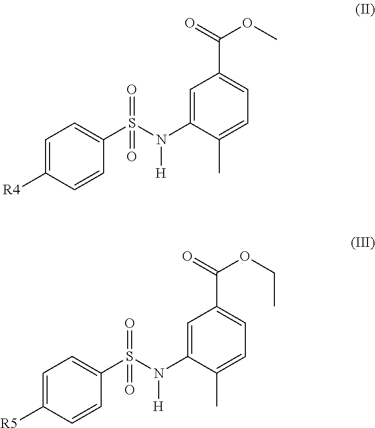
wherein
R⁴ is methyl, ethyl, n-propyl, iso-propyl, n-butyl, iso-butyl, methoxy, ethoxy, chloro, bromo, or fluoro,
R⁵ is methyl, ethyl, methoxy, ethoxy, chloro, bromo, or fluoro.

6. Composition according to claim 4 further comprising one or more cytotoxic and/or cytostatic compound.

7. Compound of formula (I), 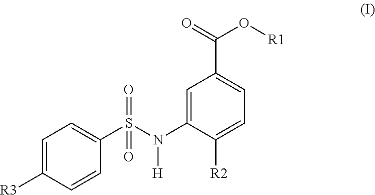
wherein
R is C₁-C₆ alkyl;
R² is H, C₁-C₆ alkyl or C₁-C₆ alkoxy; and
R³ is H, C₁-C₆ alkyl C₁-C₆ alkoxy or halogen
for use as a medicament.

8. Compound of claim 7, wherein
R¹ is C₁-C₃ alkyl; and
R² is H or C₁-C₃ alkyl.

9. Compound of claim 7, wherein
R³ is methyl, ethyl, n-propyl, iso-propyl, methoxy, ethoxy, n-propoxy, iso-propoxy, F, Cl, or Br.

10. Compound of claim 7, having a structure according to formulas (IV) to (XI)

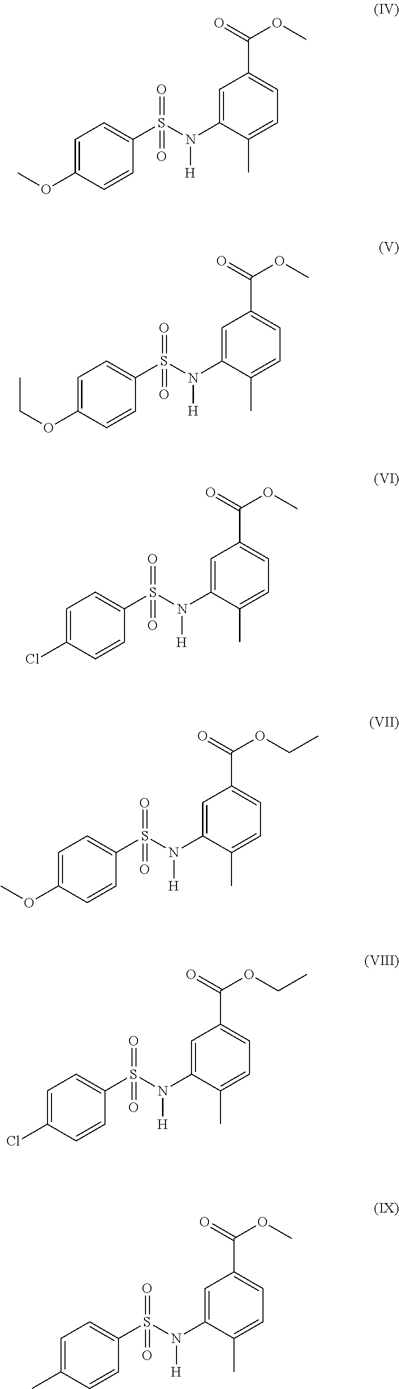

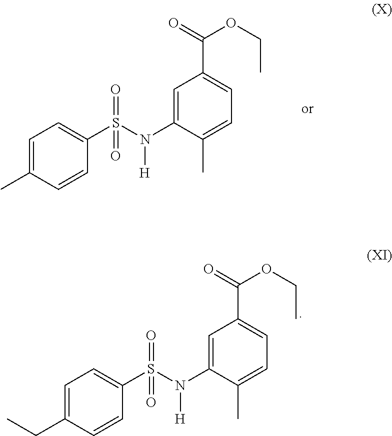

11. Compound of formula (I), 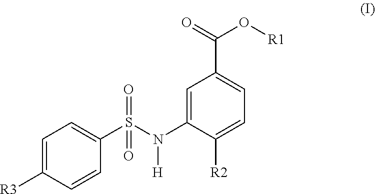
wherein
R¹ is C₁-C₆ alkyl;
R² is H, C₁-C₆ alkyl or C₁-C₆ alkoxy; and
R³ is H, C₁-C₆ alkyl C₁-C₆ alkoxy or halogen
for treating, ameliorating or preventing a hyperproliferative disease.

12. Compound of claim 11, wherein
R¹ is C₁-C₃ alkyl; and
R² is H or C₁-C₃ alkyl.

13. Compound of claim 11, wherein
R³ is methyl, ethyl, n-propyl, iso-propyl, methoxy, ethoxy, n-propoxy, iso-propoxy, F, Cl, Br or I.

14. Compound of claim 11, having a structure according to formulas (IV) to (XI).

15. Compound of claim 11, wherein the hyperproliferative disease is selected from the group consisting of precancerosis; dysplasia; metaplasia; carcinomas of the gastrointestinal or colorectal tract, liver, pancreas, kidney, bladder, prostate, endometrium, ovary, testes, melanoma, dysplastic oral mucosa, invasive oral cancers, small cell and non-small cell lung carcinomas, hormone-dependent breast cancers, hormone-independent breast cancers, transitional and squamous cell cancers, neurological malignancies including neuroblastoma, gliomas, astrocytomas, osteosarcomas, soft tissue sarcomas, hemangioamas, endocrinological tumors, hematologic neoplasias including leukemias, lymphomas, and other myeloproliferative and lymphoproliferative diseases, carcinomas in situ, hyperplastic lesions, adenomas, fibromas, histiocytosis, chronic inflammatory proliferative diseases, vascular proliferative diseases and virus-induced proliferative diseases, skin diseases characterized by hyperproliferation of keratinocytes and/or T cells.

16. Method of treating a hyperproliferative disease, wherein a pharmaceutically effective amount of a compound according to claim 1 is administered to a person in need thereof.

17. Method of claim 16, wherein the hyperproliferative disease is selected from the group consisting of precancerosis; dysplasia; metaplasia; carcinomas of the gastrointestinal or colorectal tract, liver, pancreas, kidney, bladder, prostate, endometrium, ovary, testes, melanoma, dysplastic oral mucosa, invasive oral cancers, small cell and non-small cell lung carcinomas, hormone-dependent breast cancers, hormone-independent breast cancers, transitional and squamous cell cancers, neurological malignancies including neuroblastoma, gliomas, astrocytomas, osteosarcomas, soft tissue sarcomas, hemangioamas, endocrinological tumors, hematologic neoplasias including leukemias, lymphomas, and other myeloproliferative and lymphoproliferative diseases, carcinomas in situ, hyperplastic lesions, adenomas, fibromas, histiocytosis, chronic inflammatory proliferative diseases, vascular proliferative diseases and virus-induced proliferative diseases, skin diseases characterized by hyperproliferation of keratinocytes and/or T cells.

18. Method of treating a hyperproliferative disease, wherein a pharmaceutically effective amount of a composition according to claim 4 is administered to a person in need thereof.

19. Method of treating a hyperproliferative disease, wherein a pharmaceutically effective amount of a compound according to claim 7 is administered to a person in need thereof.

20. Method of claim 18, wherein the hyperproliferative disease is selected from the group consisting of precancerosis; dysplasia; metaplasia; carcinomas of the gastrointestinal or colorectal tract, liver, pancreas, kidney, bladder, prostate, endometrium, ovary, testes, melanoma, dysplastic oral mucosa, invasive oral cancers, small cell and non-small cell lung carcinomas, hormone-dependent breast cancers, hormone-independent breast cancers, transitional and squamous cell cancers, neurological malignancies including neuroblastoma, gliomas, astrocytomas, osteosarcomas, soft tissue sarcomas, hemangioamas, endocrinological tumors, hematologic neoplasias including leukemias, lymphomas, and other myeloproliferative and lymphoproliferative diseases, carcinomas in situ, hyperplastic lesions, adenomas, fibromas, histiocytosis, chronic inflammatory proliferative diseases, vascular proliferative diseases and virus-induced proliferative diseases, skin diseases characterized by hyperproliferation of keratinocytes and/or T cells.

21. Method of claim 19, wherein the hyperproliferative disease is selected from the group consisting of precancerosis; dysplasia; metaplasia; carcinomas of the gastrointestinal or colorectal tract, liver, pancreas, kidney, bladder, prostate, endometrium, ovary, testes, melanoma, dysplastic oral mucosa, invasive oral cancers, small cell and non-small cell lung carcinomas, hormone-dependent breast cancers, hormone-independent breast cancers, transitional and squamous cell cancers, neurological malignancies including neuroblastoma, gliomas, astrocytomas, osteosarcomas, soft tissue sarcomas, hemangioamas, endocrinological tumors, hematologic neoplasias including leukemias, lymphomas, and other myeloproliferative and lymphoproliferative diseases, carcinomas in situ, hyperplastic lesions, adenomas, fibromas, histiocytosis, chronic inflammatory proliferative diseases, vascular proliferative diseases and virus-induced proliferative diseases, skin diseases characterized by hyperproliferation of keratinocytes and/or T cells.
